(19) United States
(12) Patent Application Publication
(10) Pub. No.: US 2024/0158797 A1
(43) Pub. Date: May 16, 2024

(54) ANTISENSE OLIGONUCLEOTIDES FOR THEIR USE IN AN ANTI-CANCER TREATMENT

(71) Applicants: UNIVERSITÉ GRENOBLE ALPES, Saint Martin D'Hères (FR); INSTITUT NATIONAL DE LA SANTÉ ET DE LA RECHERCHE MÉDICALE (INSERM), Paris (FR); CENTRE HOSPITALIER UNIVERSITAIRE DE GRENOBLE, La Tronche (FR)

(72) Inventors: Sandrine HUMBERT, Grenoble (FR); Fabienne AGASSE, La Tronche (FR); Frédéric SAUDOU, Grenoble (FR); Kassem RAYANE, Grenoble (FR)

(73) Assignees: UNIVERSITÉ GRENOBLE ALPES, Saint Martin D'Hères (FR); INSTITUT NATIONAL DE LA SANTÉ ET DE LA RECHERCHE MÉDICALE (INSERM), Paris (FR); CENTRE HOSPITALIER UNIVERSITAIRE DE GRENOBLE, La Tronche (FR)

(21) Appl. No.: 18/281,642

(22) PCT Filed: Mar. 18, 2022
(86) PCT No.: PCT/FR2022/050497

§ 371 Date: Sep. 12, 2023

(30) Foreign Application Priority Data
Mar. 25, 2021 (FR) 21/02992

(51) Int. Cl. C12N 15/113; A61K 31/166; A61K 31/175; A61K 31/282; A61K 31/495; A61P 35/00

(52) U.S. Cl. CPC C12N 15/1137; A61K 31/166; A61K 31/175; A61K 31/282; A61K 31/495; A61P 35/00; C12N 2310/11

(57) ABSTRACT

An antisense oligonucleotides having the ability to decrease the expression level of the huntingtin protein in glioma cells, for their use in sensitizing the cells to an anti-cancer treatment.

### TECHNICAL FIELD OF THE INVENTION

The invention relates to antisense oligonucleotides for their use in an anti-cancer treatment. It falls within the field of therapeutic applications and more particularly concerns the use of antisense oligonucleotides to sensitize glioma cells to anti-cancer treatments.

### PRIOR TECHNIQUE

The glioblastoma multiforme (GBM) or type IV astrocytoma, is a rare brain tumor (4 cases per 100,000 inhabitants in France each year), with rapid growth and whose vital prognosis is poor since only 2% of patients survive more than 2 years after diagnosis. The tumors have a glial origin, so they are called gliomas. It has been shown more recently that deregulated subventricular zone stem cells could be the cause of GBM («Human glioblastoma arises from subventricular zone cells with low-level driver mutations»Lee et al. 2018). The environmental and molecular factors at the origin of these deregulations remain to be determined. The tumors are made up of multiple more or less differentiated cell types. Among these cells, rare cancer stem cells are responsible for resistance to cancer treatments and tumor recurrence.

The first phase of treatment consists of surgical excision. However, the GBM cells proliferate rapidly and invade the cerebral parenchyma and it is therefore impossible to remove the whole tumor and in particular the stem cells. Repeated cycles of radiotherapy and chemotherapy with oral temozolomide (TMZ) for 7 days every 28 days can slow tumor regrowth. The TMZ is a DNA alkylating agent that adds a methyl group at the N-7 or O-6 position of guanine and at the N-3 position of adenine. Although the presence of methyl-06 guanine represents only less than 10% of the total alkylation due to TMZ, it is mainly through this modification that TMZ induces apoptosis. During the replication, the modified guanine mismatches with the thymine. This mismatch is recognized by the enzymes of the DNA mismatch repair system (MMR for «mismatch repair»). These enzymes loop try to repair these mismatches without success. This results in DNA double strand breaks, cell cycle arrest at G2 and induction of the apoptosis. However, recurrent tumors express the enzyme 06-methylguanine DNA methyltransferase (MGMT), an enzyme that removes the alkyl group present on guanine after treatment with TMZ. The TMZ therefore becomes ineffective and there is no treatment alternative to TMZ.

Any progress making it possible to identify molecular and cellular targets aimed at reducing the ability of tumor cells to resist to TMZ will therefore be beneficial for patients suffering from gliomas.

### Presentation of the Invention

To this end, the present invention aims to overcome the aforementioned drawbacks by providing an antisense oligonucleotide having the ability to reduce the expression level of the huntingtin protein in glioma cells, for use in sensitizing said cells to an anti-cancer treatment.

The present invention further relates to an antisense oligonucleotide having the ability to decrease the expression level of the huntingtin protein and the 6-O-methylguanine DNA methyltransferase (MGMT) protein in glioma cells, for its use in sensitizing said cells to an anti-cancer treatment.

The huntingtin protein (HTT) is a pleiotropic protein of 350 kDa. This protein is mainly studied in its mutant form, that is to say presenting an expansion of more than 36 CAG triplets in exon 1 of its gene sequence because the mutated gene (huntingtin gene with the abnormal expansion of CAG triplets) encoding the HTT protein causes Huntington's disease, a fatal neurodegenerative disease. The wild-type HTT allows maintaining the stem state of cells. Indeed, the decrease in HTT levels in stem/progenitor cells of different origins leads to a depletion of the reservoir of these cells which will tend to differentiate early (Godin et al. 2010, Elias et al. 2014, Lopes et al. 2016). The HTT has also been shown to participate in the epithelial-mesenchymal transition, and in its absence, breast cancer cells are more metastatic and non-cancerous cells lose their polarity (Elias et al. 2015, Thion et al. 2015).

The antisense oligonucleotides, also called ASO, are short synthetic DNA sequences (approximately 20 bases) which target the messenger RNA (mRNA) of interest according to the principle of Crick and Watson of nitrogenous base pairing. The mRNA is then degraded by RNAse H and is therefore not translated into protein. The ASO present phosphothiorate bonds and modifications of nitrogenous bases with in particular the addition of 2′-O-methoxyethyl (MOE) groups on the nucleotides of the 3′ and 5′ ends making them resistant to nucleases and soluble in water.

The inventors have discovered that, quite surprisingly, the ASO having the ability to reduce the expression level of the HTT protein in glioma cells by targeting their mRNA and the ASO having the ability to decrease the expression level of the HTT protein and of the MGMT protein (making the cells which express it particularly resistant to anti-cancer treatments) in glioma cells, when they are transfected to said cells, make it possible to sensitize the latter to an anti-cancer treatment to which they usually develop resistance over time.

The antisense oligonucleotide sequences used in the present invention to reduce HTT levels in glioma cells are sequences available in the literature (Kordasiewicz et al. 2012, «In Vivo Evaluation of Candidate Allele-specific Mutant Huntingtin Gene Silencing Antisense Oligonucleotides» Southwell et al. 2014). In the context of Huntington's disease, clinical trials have been carried out with the ASO of sequence SEQ ID NO: 1 also called in the description of the present application antisense oligonucleotides RG6042. After 4 intrathecal injections of ASO RG6042, HTT levels are decreased in the cerebrospinal fluid of patients with Huntington's disease (Tabrizi et al. 2019).

The 6-O-methylguanine DNA methyltransferase (MGMT) protein is a DNA repair enzyme. It is found expressed in different tissues of the body (low tissue specificity). In the brain, it is expressed by endothelial cells and glial cells but not by neurons.

The levels of MGMT are low in the brain and normally correlated with the methylation of the promoter of the gene or even of the body of the gene (mechanisms of «silencing»). In the brain tumors, following repeated cycles of radiotherapy/chemotherapy, certain cells re-expressing MGMT emerge, «take over» the other cells of the tumor mass which are partly eliminated by the treatment. These resistant cells are the cause of tumor recurrence. The use of antisense oligonucleotides having the ability to reduce the expression level of the huntingtin protein and of the 6-O-methylguanine DNA methyltransferase (MGMT) protein in glioma cells according to the invention is therefore particularly advantageous in order to avoid the phenomenon of tumor recurrence.

In particular embodiments, the invention also responds to the following characteristics, implemented separately or in each of their technically effective combinations.

In particular embodiments of the invention, the antisense oligonucleotide comprises between 12 and 35 nucleic bases.

In particular embodiments of the invention, the antisense oligonucleotide is selected from antisense oligonucleotides of sequence SEQ ID NO: 1, SEQ ID NO: 2, SEQ ID NO: 3 and SEQ ID NO: 4.

In particular embodiments of the invention, the antisense oligonucleotide is selected from antisense oligonucleotides having a sequence identical to at least 50%, preferably at least 60%, more preferably at least 70%, preferably at least 80%, preferably at least 90%, preferably at least 92%, more preferably at least 95%, preferably at least 96%, preferably at least 97%, preferably at least 98%, preferably at least 99%, with a sequence selected from SEQ ID NO: 1, SEQ ID NO: 2, SEQ ID NO: 3 and SEQ ID NO: 4.

In particular embodiments of the invention, the anti-cancer treatment to which the glioma cells are sensitized is a treatment which causes damage to the DNA of said cells.

In particular embodiments of the invention, the anti-cancer treatment to which the glioma cells are sensitized is a treatment with a DNA alkylating agent. The alkylating agent is preferably selected from temozolomide (TMZ), lomustine (CCNU), carmustine (BCNU), procarbazine and carboplatin.

In the particular embodiments of the invention in which the anti-cancer treatment to which the glioma cells are sensitized is a treatment with temozolomide (TMZ), the glioma cells are preferably sensitized so that the lethal dose 50 (LD50) at TMZ is reduced by at least 30% but preferably by at least 40%. The lethal dose 50 (LD50) designates the concentration of TMZ for which cell viability is reduced by 50% relative to the control condition (without use of antisense oligonucleotides which are the subject of the present invention).

In particular embodiments of the invention, the anti-cancer treatment to which the glioma cells are sensitized is a treatment using radiation.

According to another aspect, the invention relates to a pharmaceutical composition comprising at least one antisense oligonucleotide which is the subject of the present invention, or comprising a salt, an ester, a salt of such an ester or a pharmaceutically acceptable prodrug thereof, and a pharmaceutically acceptable carrier or diluent, for use in sensitizing said cells to anti-cancer treatment.

In this document, a «pharmaceutical composition comprising an antisense oligonucleotide» refers to a composition comprising an antisense oligonucleotide targeting huntingtin in a pharmaceutically acceptable diluent. Pharmaceutically acceptable diluents are well known to those skilled in the art. The selection of a pharmaceutically acceptable diluent or carrier is based on a number of factors, including, but not limited to, the solubility of the compound and the route of administration. These considerations are well understood by those skilled in the art.

A «pharmaceutical carrier» or excipient can be a pharmaceutically acceptable solvent, a suspending agent or any other pharmacologically inert carrier for delivering one or more nucleic acids to an animal and are known in the technical field. The excipient can be liquid or solid and is selected, taking into account the intended mode of administration, so as to obtain the volume, consistency, etc. desired when combined with a nucleic acid and the other components of a given pharmaceutical composition.

The term «pharmaceutically acceptable salt») designates the physiologically and pharmaceutically acceptable salts of the compounds of the invention: that is to say the salts which retain the desired biological activity of the parent compound and do not confer on it adverse toxicological effects. Sodium salts of antisense oligonucleotides are useful and are well accepted for therapeutic administration to humans.

The term «prodrug» herein refers to a therapeutic agent which is prepared in an inactive or less active form which is converted to an active form (that is to say a drug) in the body or cells thereof by the action of endogenous enzymes or other chemical substances and/or conditions.

According to another aspect, the present invention relates to the antisense oligonucleotide or the composition comprising at least one antisense oligonucleotide according to the present invention for its use in the treatment of a brain tumor.

According to another aspect, the present invention relates to the antisense oligonucleotide or the composition comprising at least one antisense oligonucleotide according to the present invention for its use in the treatment of glioblastoma multiforme or type IV astrocytoma.

### BRIEF DESCRIPTION OF FIGURES

The invention will be better understood on reading the following description, given by way of non-limiting example, and made with reference to the figures which represent:

FIG. 1 illustrates in A the results of the western blot carried out from cells of the U251 cell line having been transfected with the antisense oligonucleotide (ASO) RG6042 (SEQ ID NO: 1), with the ASO Scr (for «scramble») or treated with lipofectamine alone (Ctrl for control), and, in B, the quantification of HTT protein levels obtained in western blot after 4 repetitions of the experiment presented in (A);

FIG. 2 illustrates in A the result of the western blot carried out from cells of the U87 cell line having been transfected with ASO RG6042, with ASO Scr or treated with lipofectamine alone (Ctrl), and, in B, the quantification of the protein levels of HTT obtained by western blot after at least 3 repetitions of the experiment presented in (A);

FIG. 3 illustrates in A the result of the western blot carried out from cells of the T98G cell line having been transfected with ASO RG6042, with ASO Scr or treated with lipofectamine alone (Ctrl), and, in B, the quantification of the protein levels of HTT obtained in western blot after 5 repetitions of the experiment presented in (A);

FIG. 4 illustrates in A the result of the western blot carried out from cells of the T98G cell line having been transfected with ASO RG6042, with ASO Scr or treated with lipofectamine alone (Ctrl), and, in B, the quantification of the protein levels of MGMT obtained in western blot after 4 repetitions of the experiment presented in (A);

FIG. 5 illustrates the result of the western blot carried out from cells of the T98G cell line having been transfected with ASO RG6042, with ASO PH1, with ASO PH2, with ASO PH3, or with the ASO Scr;

FIG. 6 illustrates the quantification of the protein levels of HTT in A and of MGMT in B, obtained by western blot after at least four repetitions of the experiment presented in FIG. 5;

FIG. 7 illustrates a graph of HTT mRNA expression in A quantified by qPCR, and a graph of MGMT mRNA expression in B quantified by qPCR, in T98G cells treated with ASO RG6042;

FIG. 8 illustrates in A a curve showing the percentage of living cells of the U251 line transfected with ASO RG6042 or treated with lipofectamine alone (Ctrl) as a function of the dose of TMZ received and, in B, the concentration of TMZ measured at the LD50 for cells of the U251 line transfected with ASO RG6042 or treated with lipofectamine alone (Ctrl);

FIG. 9 illustrates in A a curve showing the percentage of living cells of the U87 line transfected with ASO RG6042 or treated with lipofectamine alone (Ctrl) as a function of the dose of TMZ received and, in B, the concentration of TMZ measured at the LD50 for cells of the U87 line transfected with ASO RG6042 or treated with lipofectamine alone (Ctrl);

FIG. 10 illustrates in A a curve showing the percentage of living cells of the T98G line transfected with ASO RG6042 or treated with lipofectamine alone (Ctrl) as a function of the dose of TMZ received and, in B, the concentration of TMZ measured at the LD50 for cells of the T98G line transfected with ASO RG6042 or treated with lipofectamine alone (Ctrl);

FIG. 11 illustrates in A a curve showing the percentage of living cells of the U251 line transfected with ASO RG6042 or Scr as a function of the dose of Lomustine received and, in B, the mean of the concentrations of Lomustine measured at the LD50 in 4 different MTT tests for cells of line U251 transfected with ASO RG6042 or Scr;

FIG. 12 illustrates in A a curve showing the percentage of live cells of the T98G line transfected with ASO RG6042 or Scr as a function of the dose of Lomustine received and, in B, the mean of the concentrations of Lomustine measured at the LD50 in 4 different MTT assays for T98G cell line transfected with ASO RG6042 or Scr.

### DESCRIPTION OF EMBODIMENTS

Obtaining Glioma Cells Transfected with Antisense Oligonucleotides

The U87, T98G and U251 glioma cell lines are cultured in DMEM medium («Dulbecco/Vogt modified Eagle's minimal essential medium» supplemented with 10% fetal calf serum and 1% penicillin and streptomycin.

When the cultures are at 60% confluence, they are transfected with 100 μM of antisense oligonucleotide (ASO) RG6042 (of sequence SEQ ID NO: 1), or PH1 (SEQ ID NO: 2) or PH2 (SEQ ID NO: 3) or PH3 (SEQ ID NO: 4) or Scr in lipofectamine for 48 h. The ASO Scr (for «scramble» is an ASO serving as a control whose sequence SEQ ID NO: 5 is <A*CATG*>A*C*C*G*C*A*C*T*C*A*<CTAT*A> where * is a phosphorothioate bond and the bases between the signs < and > are 2′-O-methoxyethyl RNA.

The cells are then used either in a sensitivity test to temozolomide (TMZ), or in a sensitivity test to the alkylating agent Lomustine, or in huntingtin (HTT) and 6-O-methylguanine DNA methyltransferase (MGMT) protein level expression tests.

Huntingtin (HTT) Protein Expression Level Test

Cells transfected with the ASO RG6042, the ASO Scr, or treated with lipofectamine alone (for control), are harvested for testing the expression level of the HTT protein.

The cells are rinsed with phosphate-buffered saline (PBS) and then lysed in RIPA-type lysis buffer supplemented with protease and phosphatase inhibitors. The proteins solubilized in the buffer are harvested after centrifugation, denatured in Laemmli loading buffer and deposited on polyacrylamide gel. After migration, the proteins are transferred onto a polyvinylidine difluoride (PVDF) membrane. The HTT protein is revealed by incubation with the D7F7 antibody (#5656, from Cell signaling Technology®). The vinculin is used as a load control.

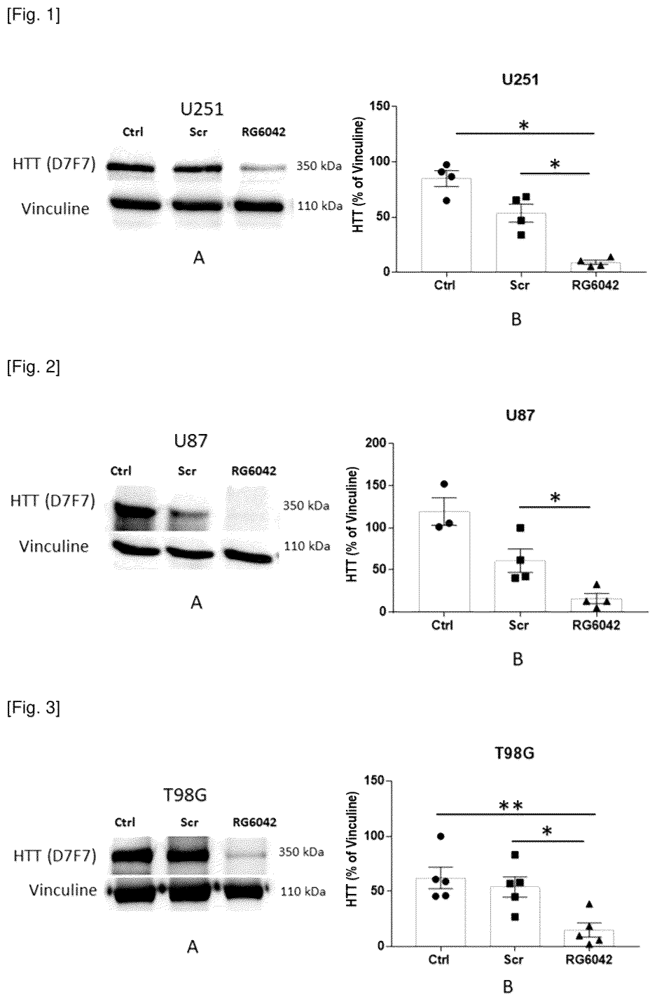

FIG. 1 illustrates in A the results of the western blot carried out from cells of the U251 cell line having been transfected with ASO RG6042, with the ASO Scr or simply treated with lipofectamine alone (Ctrl for control), and, in B, the quantification of HTT protein levels obtained by western blot after 4 repetitions of the experiment presented in A.

FIG. 2 illustrates in A the result of the western blot produced from cells of the U87 cell line having been transfected with ASO RG6042, with ASO Scr or treated with lipofectamine alone (Ctrl), and, in B, the quantification of HTT protein levels obtained by western blot after at least 3 repetitions of the experiment presented in A.

FIG. 3 illustrates in A the result of the western blot produced from cells of the T98G cell line having been transfected with ASO RG6042, with ASO Scr or treated with lipofectamine alone (Ctrl), and, in B, the quantification of HTT protein levels obtained by western blot after 5 repetitions of the experiment presented in A.

This test shows that ASO RG6042 significantly reduces HTT protein levels in glioma cells.

Test of the Expression Level of the 6-O-Methylguanine DNA Methyltransferase (MGMT) Protein

Cells transfected with ASO RG6042, ASO Scr, or treated with lipofectamine alone (for control), are harvested for the test of the expression level of the MGMT protein.

The protocol is the same as for the previously cited test of the expression level of the HTT protein with the only change being the use of anti-MGMT antibodies (#2739, from the Cell signaling Technology® brand) in incubation to reveal the MGMT protein. The vinculin is also used here as a load control.

FIG. 4 illustrates in A the result of the western blot produced from cells of the T98G cell line having been transfected with ASO RG6042, with ASO Scr or treated with lipofectamine alone (Ctrl) and, in B, the quantification of MGMT protein levels obtained by western blot after at least 4 repetitions of the experiment presented in A.

This test shows that ASO RG6042 significantly reduces MGMT levels in the T98G cells.

Test of the Expression Level of the Huntingtin (HTT) Protein and the 6-O-Methylguanine DNA Methyltransferase (MGMT) Protein in the T98G Cells

T98G cells transfected with ASO RG6042, or ASO PH1, or ASO PH2 or ASO PH3, or ASO Scr, are harvested for testing the expression level of HTT proteins and MGMT.

The protocol is the same as for the previously cited tests for the expression level of the HTT protein and the expression level of the MGMT protein. The vinculin is also used here as a load control.

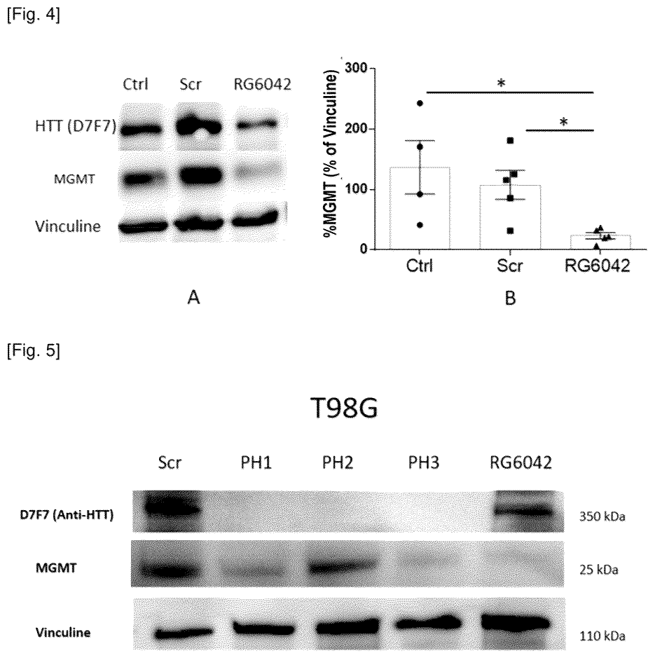

FIG. 5 illustrates the result of the western blot produced from cells of the T98G cell line having been transfected with ASO RG6042, with ASO PH1, with ASO PH2, with ASO PH3, or with ASO Scr.

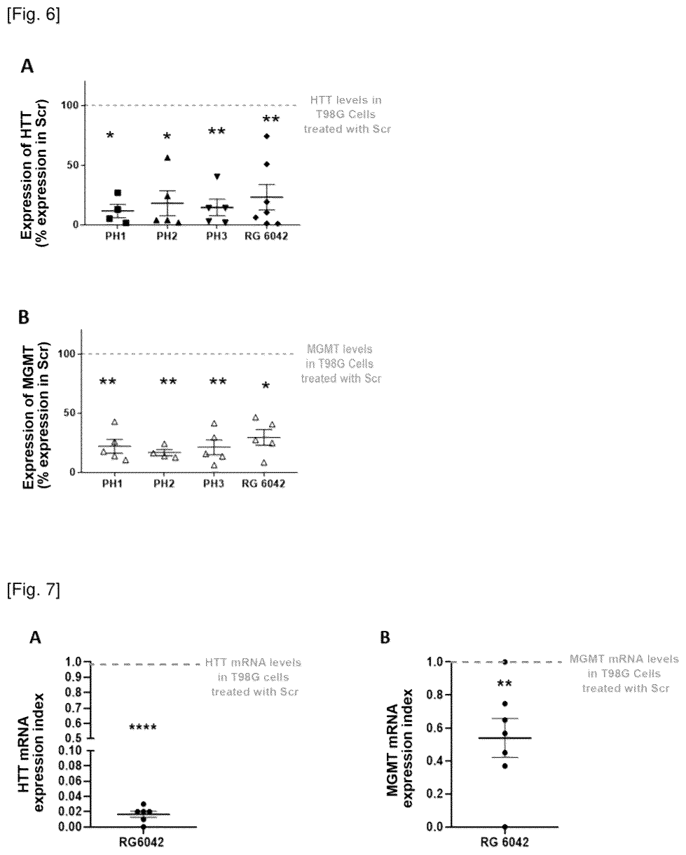

FIG. 6 illustrates the quantification of the protein levels of HTT in A and of MGMT in B, obtained by western blot after at least four repetitions of the experiment presented in FIG. 5. The statistics were carried out using the Kruskal-wallis test followed by Dunn's multiple comparison test, error bars, SEM, *p<0.05, **p<0.001.

FIG. 7 illustrates the expression of the mRNA of HTT in A and of MGMT in B quantified by qPCR. Statistics were established using Mann Whitney's T-test, error bars, SEM, *p<0.05, ****p<0.0001.

This test shows that the ASO RG6042, PH1, PH2 and PH3 significantly reduce the levels of HTT and MGMT protein in the T98G glioma cells.

Temozolomide (TMZ) Sensitivity Test

The cells transfected with ASO RG6042 or treated with lipofectamine alone (Ctrl for control) are harvested for the TMZ sensitivity test.

The TMZ was prepared in the form of a stock solution of 50 mM in 33.3 mM of citrate monohydrate. The solution is sonicated, filtered and frozen at −20° C. until further use (Nygren, H., and Eksborg, S. (2012), «Stability of temozolomide in solutions aimed for oral treatment prepared from a commercially available powder for infusion», Pharm Methods 3,1-3).

The cells are distributed in 96-well culture dishes at the rate of 200 cells per well for the U251 and T98G lines and 100 cells per well for the U87 line. The cells are left developing for 24 hours.

The cells are then treated for 48 hours with increasing doses of TMZ at the rate of 10 μM to 110 μM for the U87 and U251 lines and from 100 μM to 1500 μM for the T98G line. Each dilution is tested in triplicate. The control condition consists of cells treated with complete culture medium. The blank wells contain only complete culture medium.

At the end of the 48 hours of treatment with TMZ, the medium containing the TMZ is removed and replaced with fresh complete culture medium in which the cells are left to incubate for 72 hours.

An MTT test (known live cell count test) is then set up in order to evaluate the cell survival in the different wells.

The yellow probe of MTT, 3-(4,5-Dimethyl-2-thiazolyl)-2,5-diphenyl-2H-tetrazolium bromide, is then added at a working concentration of 0.5 mg·ml⁻¹ to each well. The wells are incubated for 4 hours in an incubator humidified with 95% air and 5% CO₂ at 37° C. The insoluble purple formazan resulting from the reduction of the MTT probe in living cells is solubilized by adding a solubilizing solution (10% sodium dodecyl sulfate (SDS) and 0.01% hydrogen chloride (HCl) in water) overnight at 37° C. The optical density (OD) of the contents of each well is then analyzed with a spectrophotometer at wavelengths comprised between 490 nm and 570 nm.

The lethal dose 50 (LD50) denotes the concentration of TMZ for which cell viability is reduced by 50% relative to the control condition.

FIG. 8 illustrates in A a curve showing the percentage of live cells of the U251 line transfected with ASO RG6042 or treated with lipofectamine alone (Ctrl) as a function of the received dose of TMZ and, in B, the average TMZ concentrations measured at the LD50 in 4 different MTT tests for cells of the U251 line transfected with ASO RG6042 or treated with lipofectamine alone (Ctrl).

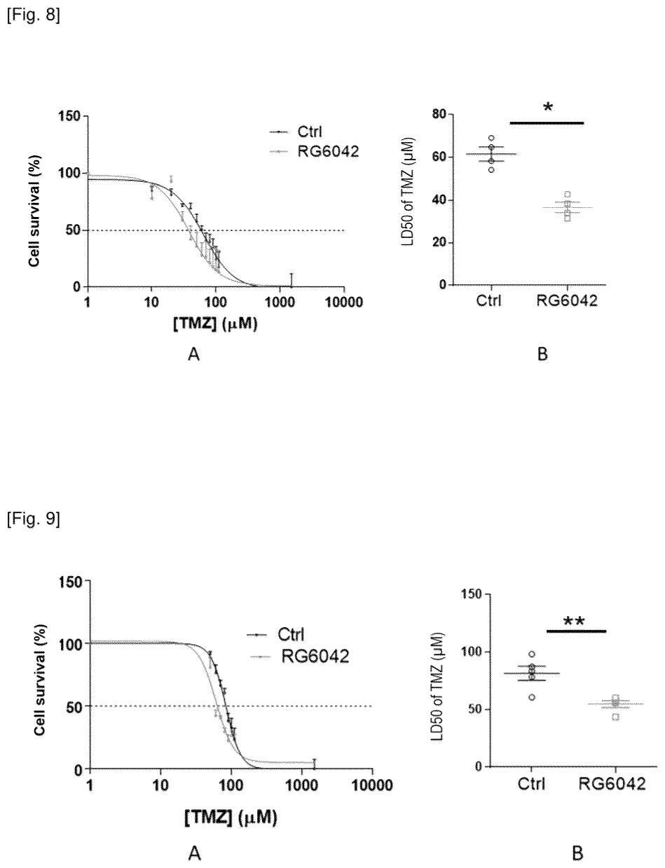

FIG. 9 illustrates in A a curve showing the percentage of living cells of the U87 line transfected with ASO RG6042 or treated with lipofectamine alone (Ctrl) as a function of the received dose of TMZ and, in B, the averages TMZ concentrations measured at the LD50 in 4 different MTT tests for cells of the U251 line transfected with ASO RG6042 or treated with lipofectamine alone (Ctrl).

FIG. 10 illustrates in A a curve showing the percentage of living cells of the T98G line transfected with ASO RG6042 or treated with lipofectamine alone (Ctrl) as a function of the received dose of TMZ and, in B, the average TMZ concentrations measured at the LD50 in 6 different MTT tests for cells of the T98G line transfected with ASO RG6042 or treated with lipofectamine alone (Ctrl).

It therefore appears that the LD50 at TMZ of the three cell lines U87, U251 and T98G is reduced in the cultures exhibiting a reduced level of HTT.

The reduction in the level of HTT leads to a reduction in the LD50 at the TMZ of:
32% in U87 cultures (LD50=81.7 t 6.2 μM against DL50=55.0 t 3.0 μM after treatment with RG6042),
40% in cultures of U251 (DL50=61.6±3.3 μM against DL50=36.7±2.6 μM after treatment with RG6042),
45% in T98G cultures (LD50=968.1±63.0 μM against LD50=531.6±51.6 μM after treatment with RG6042).

Sensitivity Test to the Alkylating Agent Lomustine

The cells transfected with ASO RG6042 or ASO Scr are harvested for the sensitivity test to the alkylating agent Lomustine.

The Lomustine was prepared in the form of a stock solution of 50 mg/ml (214 mM) in pure ethanol. The solution was filtered to sterilize it (0.2 μm pore filter) and frozen at −20° C. until further use.

The cells are distributed in 96-well culture dishes at the rate of 200 cells per well for the U251 and T98G lines. The cells are left developing for 24 hours.

The cells are then treated for 48 hours with increasing doses of Lomustine at the rate of 5 μM to 500 μM for the U251 line and from 100 μM to 1500 μM for the T98G line. Each dilution is tested in triplicate. The control condition consists of cells treated with complete culture medium. Blank wells contain only complete culture medium.

At the end of the 48h treatment with Lomustine, the medium containing Lomustine is removed and replaced with fresh complete culture medium in which the cells are left to incubate for 72h.

An MTT test is then set up in the same way as the previous test concerning sensitivity to TMZ in order to evaluate the cell survival in the different wells.

The lethal dose 50 (LD50) denotes the concentration of Lomustine for which cell viability is reduced by 50% relative to the control condition.

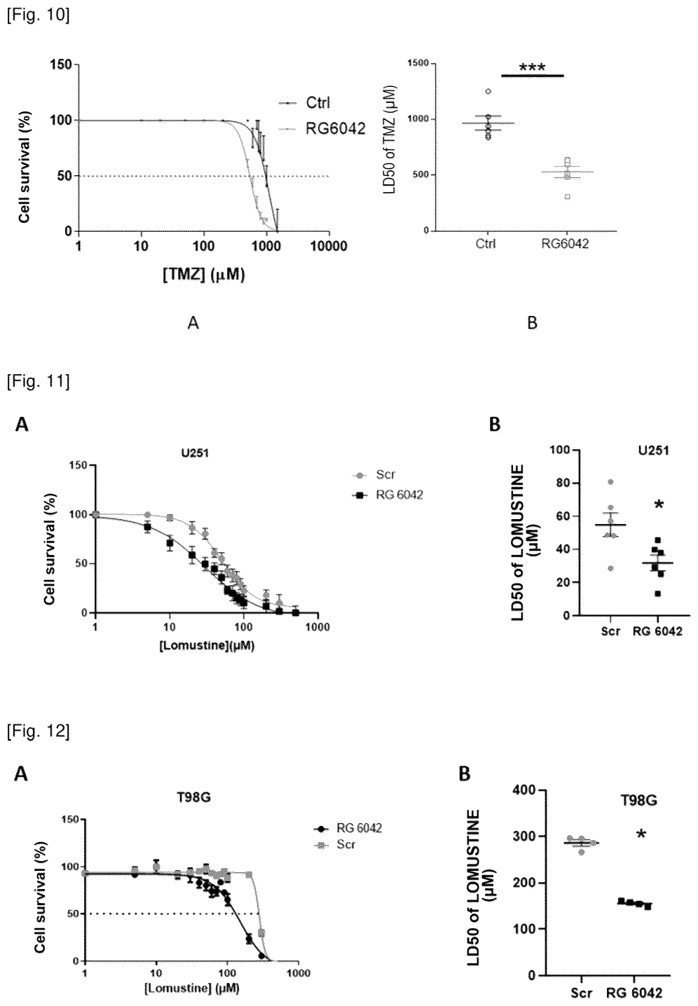

FIG. 11 illustrates in A a curve showing the percentage of living cells of the U251 line transfected with ASO RG6042 or Scr as a function of the dose of Lomustine received and, in B, the mean of the concentrations of Lomustine measured at the LD50 in 4 different MTT assays for U251 cell line transfected with ASO RG6042 or Scr.

FIG. 12 illustrates in A a curve showing the percentage of live cells of the T98G line transfected with ASO RG6042 or Scr as a function of the received dose of Lomustine and, in B, the mean of the concentrations of Lomustine measured at the LD50 in 4 different MTT assays for T98G cell line transfected with ASO RG6042 or Scr.

It therefore appears that the Lomustine LD50 of the U251 and T98G cell lines is reduced in the cultures exhibiting a reduced level of HTT.

The reduction in the level of HTT leads to a reduction in the LD50 of Lomustine by:
41% in cultures of U251 (LD50=66.79 t 4.9 μM against LD50=39.64 t 2.08 μM after treatment with RG6042);
47% in T98G cultures (LD50=279.7±7.09 μM against LD50=149.9±2.85 μM after treatment with RG6042).

The reduction in HTT levels by ASO RG6042 therefore sensitizes the U251 and T98G cells to the alkylating agent Lomustine.

More generally, it should be noted that the embodiments of the invention considered above have been described by way of non-limiting examples and that other variants are therefore possible.

1. An antisense oligonucleotide having the ability to reduce the expression level of the huntingtin protein in glioma cells, for its use in sensitizing the cells to an anti-cancer treatment, the antisense oligonucleotide being selected from the antisense oligonucleotides of sequence SEQ ID NO: 1, SEQ ID NO: 2, SEQ ID NO: 3 and SEQ ID NO: 4 and the antisense oligonucleotides having a sequence identical to at least 50%, with a sequence selected from SEQ ID NO: 1, SEQ ID NO: 2, SEQ ID NO: 3 and SEQ ID NO: 4.

2. The antisense oligonucleotide for its use according to claim 1, the antisense oligonucleotide further having the ability to decrease the expression level of the protein 6-O-methylguanine DNA methyltransferase (MGMT) in glioma cells.

3. The antisense oligonucleotide for its use according to claim 1, the antisense oligonucleotide comprising between 12 and 35 nucleic bases.

4. The antisense oligonucleotide for its use according to claim 1, wherein the anti-cancer treatment to which the glioma cells are sensitized is a treatment which causes damage to the DNA of the cells.

5. The antisense oligonucleotide for its use according to claim 1, wherein the anti-cancer treatment to which the glioma cells are sensitized is a treatment with a DNA alkylating agent.

6. The antisense oligonucleotide for its use according to claim 1, wherein the anti-cancer treatment to which the glioma cells are sensitized is a treatment with one or a combination of several of the alkylating agents selected from temozolomide (TMZ), lomustine (CCNU), carmustine (BCNU), procarbazine and carboplatin.

7. The antisense oligonucleotide for its use according to claim 1, wherein the anti-cancer treatment to which the glioma cells are sensitized is a temozolomide (TMZ) treatment and the glioma cells are sensitized such that the lethal dose 50 (LD50) at TMZ is reduced by at least 30%.

8. The antisense oligonucleotide for its use according to claim 1, wherein the anti-cancer treatment to which the glioma cells are sensitized is a treatment using radiation.

9. A pharmaceutical composition comprising at least one antisense oligonucleotide for its use according to claim 1, or comprising a salt, an ester, a salt of such an ester or a pharmaceutically acceptable prodrug thereof, and a pharmaceutically acceptable carrier or diluent.

10. The antisense oligonucleotide according to claim 1, for its use in the treatment of a brain tumor.

11. The antisense oligonucleotide according to claim 1, for its use in the treatment of glioblastoma multiforme or type IV astrocytoma.


SEQUENCE LISTING (5 sequences)

SEQ ID NO: 1 - DNA, 20 bp, ARTIFICIAL SEQUENCE
  ANTISENSE OLIGONUCLEOTIDE FOR TARGETING HUMAN HTT RNA
ctcagtaacattgacaccac

SEQ ID NO: 2 - DNA, 20 bp, ARTIFICIAL SEQUENCE
  ANTISENSE OLIGONUCLEOTIDE TARGETING HUMAN HTT RNA
tctctattgcacattccaag

SEQ ID NO: 3 - DNA, 20 bp, ARTIFICIAL SEQUENCE
  ANTISENSE OLIGONUCLEOTIDE TARGETING HUMAN HTT RNA
ctcgactaaagcaggatttc

SEQ ID NO: 4 - DNA, 20 bp, ARTIFICIAL SEQUENCE
  ANTISENSE OLIGONUCLEOTIDE TARGETING HUMAN HTT RNA
cctgcatcagctttatttgt

SEQ ID NO: 5 - DNA, 20 bp, ARTIFICIAL SEQUENCE
  ANTISENSE OLIGONUCLEOTIDE USED AS CONTROL IN EXPERIMENTS
  misc_feature (1)..(5): 2′-O-methoxyethyl RNA sequence
  misc_feature (1)..(2): phosphorothioate bond
  misc_feature (5)..(16): phosphorothioate bonds
  misc_feature (6)..(15): DNA SEQUENCE
  misc_feature (16)..(20): 2′-O-methoxyethyl RNA sequence
  misc_feature (19)..(20): phosphorothioate bond
acatgaccgcactcactata